(19) United States
(12) Patent Application Publication
(10) Pub. No.: US 2014/0045847 A1
(43) Pub. Date: Feb. 13, 2014

(54) CRYSTALLINE FORM OF A SALT OF A MORPHOLINO SULFONYL INDOLE DERIVATIVE AND A PROCESS FOR ITS PREPARATION

(71) Applicants: Suneel Manohar Babu Chennamsetty, Mumbai (IN); Kishor Joshi, Mumbai (IN); Yogesh Chinchwade, Mumbai (IN); Yogesh Hulawale, Mumbai (IN); Selvam Paramasivan, Mumbai (IN); Meenakshi Sivakumar, Mumbai (IN); Sivaramakrishnan Hariharan, Mumbai (IN)

(72) Inventors: Suneel Manohar Babu Chennamsetty, Mumbai (IN); Kishor Joshi, Mumbai (IN); Yogesh Chinchwade, Mumbai (IN); Yogesh Hulawale, Mumbai (IN); Selvam Paramasivan, Mumbai (IN); Meenakshi Sivakumar, Mumbai (IN); Sivaramakrishnan Hariharan, Mumbai (IN)

(73) Assignee: PIRAMAL ENTERPRISES LIMITED, Mumbai (IN)

(21) Appl. No.: 14/112,671

(22) PCT Filed: Apr. 19, 2012
(86) PCT No.: PCT/IB12/51967

§ 371 Date: Oct. 18, 2013

Related U.S. Application Data
(60) Provisional application No. 61/477,937, filed on Apr. 21, 2011.

(51) Int. Cl. C07D 413/14; A61K 45/06; A61K 31/5377

(52) U.S. Cl. CPC C07D 413/14; A61K 31/5377; A61K 45/06
USPC 514/235.2; 544/130

(57) ABSTRACT

The present invention relates to a crystalline form of a pharmaceutically acceptable salt of a morpholino sulphonyl indole derivative, that is capable of inhibiting, modulating and/or regulating Insulin-Like-Growth Factor I Receptor and Insulin Receptor. The present invention also relates to a process for the preparation of the crystalline form of said compound. Formula (I):

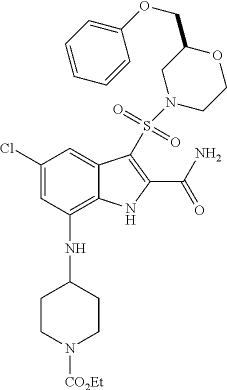 [SMILES: CCOC(=O)N1CCC(Nc2cc(Cl)cc3c(S(=O)(=O)N4CCO[C@H](COc5ccccc5)C4)c(C(N)=O)[nH]c23)CC1]

### FIELD OF THE INVENTION

The present invention relates to a crystalline form of a pharmaceutically acceptable salt of a morpholino sulfonyl indole derivative (as described herein) that is capable of inhibiting, modulating and/or regulating Insulin-Like-Growth Factor I Receptor (IGF-1R) and Insulin Receptor (IR); and a process for its preparation.

### BACKGROUND OF THE INVENTION

Protein kinases (PKs) are enzymes that catalyze the phosphorylation of hydroxy groups on tyrosine, serine and threonine residues of proteins. The consequences of this seemingly simple activity are staggering; cell growth, differentiation and proliferation; i.e., virtually all aspects of cell life, in one way or another depend on PK activity. Furthermore, abnormal PK activity has been related to a host of disorders, ranging from relatively non life-threatening diseases such as psoriasis to extremely virulent diseases such as glioblastoma (brain cancer). PKs can be broken into two classes, the protein tyrosine kinases (PTKs) and the serine-threonine kinases (STKs).

Certain growth factor receptors exhibiting PK activity are known as receptor tyrosine kinases (RTKs). They comprise a large family of transmembrane receptors with diverse biological activity. At present, at least nineteen (19) distinct subfamilies of RTKs have been identified. One RTK subfamily contains the insulin receptor (IR), insulin-like growth factor I receptor (IGF-1R) and insulin receptor related receptor (IRR). IR and IGF-1R interact with insulin to activate a hetero-tetramer composed of two entirely extracellular glycosylated a subunits and two β subunits which cross the cell membrane and which contain the tyrosine kinase domain. The Insulin-like Growth Factor-1 Receptor (IGF-1R), and its ligands, IGF-1 and IGF-2, are abnormally expressed in numerous tumors, including, but not limited to, breast, prostate, thyroid, lung, hepatoma, colon, brain, neuroendocrine, and others.

Numerous IGF-1R small molecule inhibitors have been found to inhibit cancer growth in vitro, in vivo and in clinical trials. For example, BMS-754807 effectively inhibits the growth of a broad range of human tumor types in vitro, including mesenchymal (Ewing's, rhabdomyosarcoma, neuroblastoma, and liposarcoma), epothelial (breast, lung, pancreatic, colon, gastric), and hematopoietic (multiple myeloma and leukemia) tumor cell lines. Carboni et al., Mol Cancer Ther 2009; 8(12).

The association between abnormal PK activity and disease is not restricted to cancer. For example, RTKs have been associated with diseases such as psoriasis, diabetes mellitus, endometriosis, angiogenesis, atheromatous plaque development, Alzheimer's disease, epidermal hyperproliferation, neurodegenerative diseases, age-related macular degeneration and hemangiomas. Defects in Insulin-R and IGF-1R are indicated in type-II diabetes mellitus. A more complete correlation between specific RTKs and their therapeutic indications is set forth in Plowman et al., DN&P, 1994, 7:334-339.

The amorphous or non-crystalline form of a pharmaceutically acceptable salt, in particular, methane sulfonate salt of the morpholino sulfonyl indole derivative, (S)-ethyl 4-(2-carbamoyl-5-chloro-3-(2-(phenoxymethyl)morpholinosulfonyl)-1H-indol-7-ylamino)piperidine-1-carboxylate, that is capable of inhibiting, modulating and/or regulating Insulin-Like-Growth Factor I Receptor and Insulin Receptor has been disclosed in the applicant's co-pending PCT patent application. The amorphous or non-crystalline form had relatively inadequate shelf-life due to stability problems under stress conditions, which caused difficulty in reproducing its the pharmacological activity. Therefore, there was a need for developing a process for the preparation of a stable crystalline form of said derivative for overcoming the problems associated with the instability of the amorphous or non-crystalline form of the specified compound, which problems have been addressed by the applicant in the current patent application by providing a stable crystalline form of the compound, designated herein as the Compound I.

### SUMMARY OF THE INVENTION

In one aspect, the present invention relates to a crystalline form of a pharmaceutically acceptable salt of a morpholino sulfonyl indole derivative, particularly, (S)-ethyl 4-(2-carbamoyl-5-chloro-3-(2-(phenoxymethyl)morpholinosulfonyl)-1H-indol-7-ylamino) piperidine-1-carboxylate methane sulfonate (herein after referred to as Compound I).

In another aspect, the present invention relates to a process for the preparation of the crystalline form of Compound I.

In yet another aspect, the present invention relates to a pharmaceutical composition comprising a therapeutically effective amount of the crystalline form of Compound I and one or more pharmaceutically acceptable excipients or carriers.

In a further aspect, the present invention relates to a pharmaceutical composition comprising a therapeutically effective amount of the crystalline form of Compound I, and a pharmaceutically acceptable carrier and optionally other therapeutic agents.

In another aspect, the present invention relates to a crystalline form of the Compound I for use in the treatment of an Insulin-Like-Growth Factor I Receptor (IGF-1R) or Insulin Receptor (IR) mediated disease or disorder by administering to a subject in need thereof, a therapeutically effective amount of the crystalline form of Compound I.

In yet another aspect, the present invention relates to a crystalline form of the Compound I for use in the treatment of cancer, by administering to a subject in need thereof, a therapeutically effective amount of the crystalline form of Compound I.

In another aspect, the present invention relates to a method for the treatment of an Insulin-Like-Growth Factor I Receptor (IGF-1R) or Insulin Receptor (IR) mediated disease or disorder by administering to a subject in need thereof, a therapeutically effective amount of the crystalline form of Compound I.

In yet another aspect, the present invention relates to a method of treatment of cancer by administering to a subject in need thereof, a therapeutically effective amount of the crystalline form of Compound I.

In a further aspect, the present invention relates to use of the crystalline form of Compound I for the treatment of an Insulin-Like-Growth Factor I Receptor (IGF-1R) or Insulin Receptor (IR) mediated disease or disorder.

In a still further aspect, the present invention relates to use of the crystalline form of Compound I for the treatment of cancer.

In another aspect, the present invention relates to use of the crystalline form of Compound I for the manufacture of a medicament for use in the treatment of an Insulin-Like-Growth Factor I Receptor (IGF-1R) or Insulin Receptor (IR) mediated disease or disorder.

In yet another aspect, the present invention relates to use of the crystalline form of Compound I for the manufacture of a medicament for use in the treatment of cancer.

### BRIEF DESCRIPTION OF THE DRAWINGS

FIG. 1 shows characteristic X-Ray powder diffraction spectrum (diffractogram) of the crystalline form of Compound I obtained when solvent used for crystallization is isopropyl acetate.

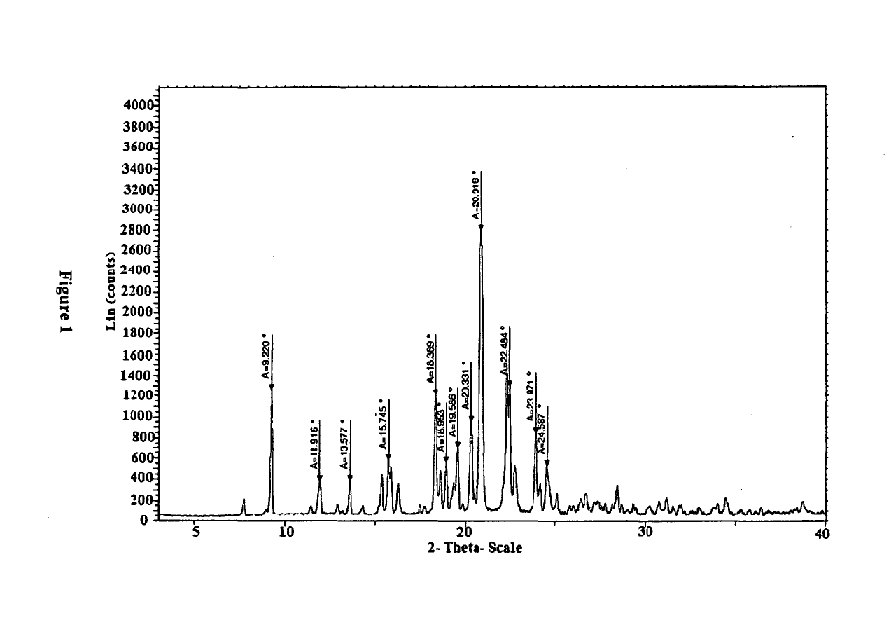

FIG. 2 shows characteristic differential scanning calorimetric (DSC) thermogram for the crystalline form of Compound I obtained when solvent used for crystallization is isopropyl acetate.

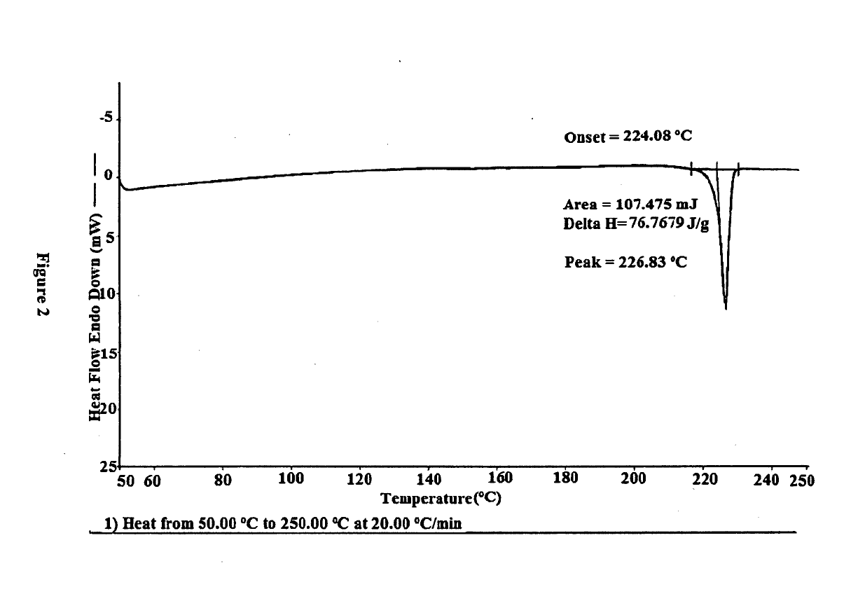

### DETAILED DESCRIPTION OF THE INVENTION

The present invention provides a crystalline form of a pharmaceutically acceptable salt of a morpholino sulfonyl indole derivative, particularly, (S)-ethyl 4-(2-carbamoyl-5-chloro-3-(2-(phenoxymethyl)morpholinosulfonyl)-1H-indol-7-ylamino)piperidine-1-carboxylate methane sulfonate (Compound I) and a process for its preparation.

The crystalline form of compound I is useful in the inhibition of Insulin-Like-Growth Factor I Receptor (IGF-1R) and Insulin Receptor (IR).

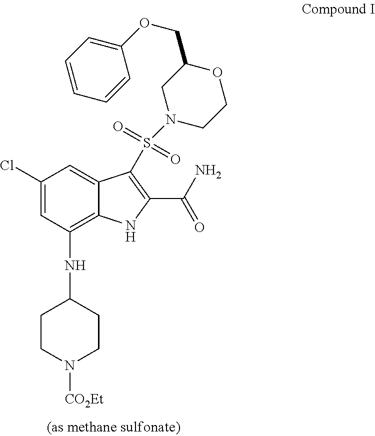 [SMILES: CCOC(=O)N1CCC(Nc2cc(Cl)cc3c(S(=O)(=O)N4CCO[C@H](COc5ccccc5)C4)c(C(N)=O)[nH]c23)CC1]

Unless otherwise indicated, the term “compound I” as used herein and the appended claims refers to, (S)-ethyl 4-(2-carbamoyl-5-chloro-3-(2-(phenoxymethyl)morpholinosulfonyl)-1H-indol-7-ylamino)piperidine-1-carboxylate methane sulfonate. However, the free base namely (S)-ethyl 4-(2-carbamoyl-5-chloro-3-(2-(phenoxymethyl)morpholinosulfonyl)-1H-indol-7-ylamino)piperidine-1-carboxylic acid is referred to herein as the free base of compound I or compound I free base.

The term “amorphous form of compound I” encompasses within its scope, the amorphous form of compound I or its mixture with one or more crystalline form(s) of compound I.

Many pharmaceutically active compounds have been found to exist in more than one polymorphic form, such as one or more crystalline forms, an amorphous form, and/or sometimes one or more solvated forms. Frequently it is found that the different forms have different physical or chemical properties, such as solubility, hygroscopicity, etc., or have properties that render some form easier to formulate into a pharmaceutical product. In addition, certain forms can have a greater stability than the other forms, as shown by a decreased tendency to spontaneously convert into a different polymorphic form or to entrap impurity causing instability. Unfortunately, predicting the potential number, or even the existence, of polymorphs for a given molecule is not possible. However, regulatory agencies desire that the various polymorphic forms of a compound be identified before a pharmaceutical product is approved for marketing, because it is essential that a product will remain stable and have predictable properties during its entire shelf life.

The preparation of the amorphous form of Compound I has been described in a co-pending patent application of the applicant. The amorphous form of Compound I ((S)-ethyl 4-(2-carbamoyl-5-chloro-3-(2-(phenoxymethyl)morpholinosulfonyl)-1H-indol-7-ylamino)piperidine-1-carboxylate methane sulfonate) was found to be unstable under stress conditions, due to its tendency to entrap methane sulfonic acid used for the salt preparation. The said compound I in its amorphous form was found to have a relatively inadequate shelf life due to a slow rate of degradation caused by an entrapped acid, which caused difficulty in reproducing its pharmacological activity. Therefore, there was a need for developing a process for preparation of a stable form of the Compound I with a view to obtain reproducibility of the compound's pharmacological activity. The synthesis provided in the current invention affords a crystalline form of Compound I, which is stable with reproducible pharmacological activity even under stress conditions or after elapse of long duration of time. The current synthesis facilitates a large-scale or commercial synthesis by incorporating a sequence of techniques known in the art, as well as the methods set forth below, from readily available starting materials.

Polymorphs of compounds are generally prepared by crystallization of the compounds under different conditions. The different conditions are, for example, using different commonly used solvents or their mixtures for crystallization; crystallization at different temperatures; various modes of cooling, ranging from very fast to very slow cooling during crystallizations. Polymorphs can also be obtained by heating or melting the compound followed by gradual or fast cooling. The presence of polymorphs can be determined by IR (Infra-red) spectroscopy, solid probe NMR (Nuclear Magnetic Resonance) spectroscopy, differential scanning calorimetry, powder X-ray diffraction or such other techniques. In an embodiment, the present invention relates to a process for the preparation of the crystalline form of Compound I, comprising:

Step 1) purifying the free base of Compound I by treating a solution of said free base in a solvent selected from isopropyl acetate, THF, 2-methyl tetrahydrofuran, toluene, heptane, methylethylketone, ethyl acetate, isopropyl acetate or combinations thereof; with activated charcoal and Si-thiol (silicycle), filtering the resulting mixture through a celite bed, repeatedly washing the celite bed with the same solvent and evaporation of the filtrate; and
Step 2) reacting the purified free base of compound I with methanesulfonic acid in said solvent at a temperature range of 70-80° C. for about 4-5 h to initiate crystallization of the Compound I as methane sulfonate salt, followed by cooling the resulting reaction mixture to room temperature and further to a temperature range of 0-5° C. to afford a crystalline mass, which is optionally washed with the same chilled solvent to obtain the required crystalline form of Compound I.

In an embodiment, the solvent used in step 1) and step 2) above may be selected from THF, 2-methyl tetrahydrofuran, a mixture of 2-methyl tetrahydrofuran and toluene, a mixture of 2-methyl tetrahydrofuran and heptane, methylethylketone, ethyl acetate or isopropyl acetate.

In a further embodiment, the solvent used in step 1) and step 2) above is isopropyl acetate.

It has been found that the crystalline form of the compound I obtained with each of the above-mentioned solvents is the same.

In another embodiment, the present invention relates to a process for the preparation of the crystalline form of Compound I, comprising crystallizing the amorphous form of the compound I((S)-ethyl 4-(2-carbamoyl-5-chloro-3-(2-(phenoxymethyl)morpholinosulfonyl)-1H-indol-7-ylamino)piperidine-1-carboxylate methane sulfonate) with a solvent selected from isopropyl acetate, THF, 2-methyl tetrahydrofuran, toluene, heptane, methylethylketone, ethyl acetate, isopropyl acetate or combinations thereof. The amorphous form of the compound I is obtained by reacting the free base of Compound I with methanesulphonic acid in THF as the solvent at room temperature for about 30 min to 2 h, according to the process for preparation of the amorphous form of compound I, as disclosed by the applicant in a co-pending PCT patent application.

In an embodiment, the solvent used for crystallization of the amorphous form of the compound I may be selected from THF, 2-methyl tetrahydrofuran, a mixture of 2-methyl tetrahydrofuran and toluene, a mixture of 2-methyl tetrahydrofuran and heptane, methylethylketone, ethyl acetate or isopropyl acetate.

In another embodiment, the solvent used for crystallization of the amorphous form of the compound I is isopropyl acetate.

In an embodiment, the crystalline form of the Compound I of the present invention is characterized by X-Ray diffraction peaks at an angle of refraction 2-theta of 9.22, 11.92, 13.58, 15.74, 18.37, 18.65, 18.95, 19.37, 19.59, 20.33, 20.92, 22.48, 22.79, 23.97, 24.19, 24.59, 28.48±0.2°.

In another embodiment, the crystalline form of the Compound I of the present invention is characterized by the melting temperature onset of the crystalline form of Compound I was determined by differential scanning calorimetry (DSC) which is found to be 224.08±0.5° C. at 20 deg/min under nitrogen, with a peak melting temperature of 226.83±0.5° C.

According to the present invention, process for the preparation of the free base of the compound I from which the crystalline form of Compound I (as methane sulfonate salt) is prepared, employs reaction steps as shown in the following scheme 1.

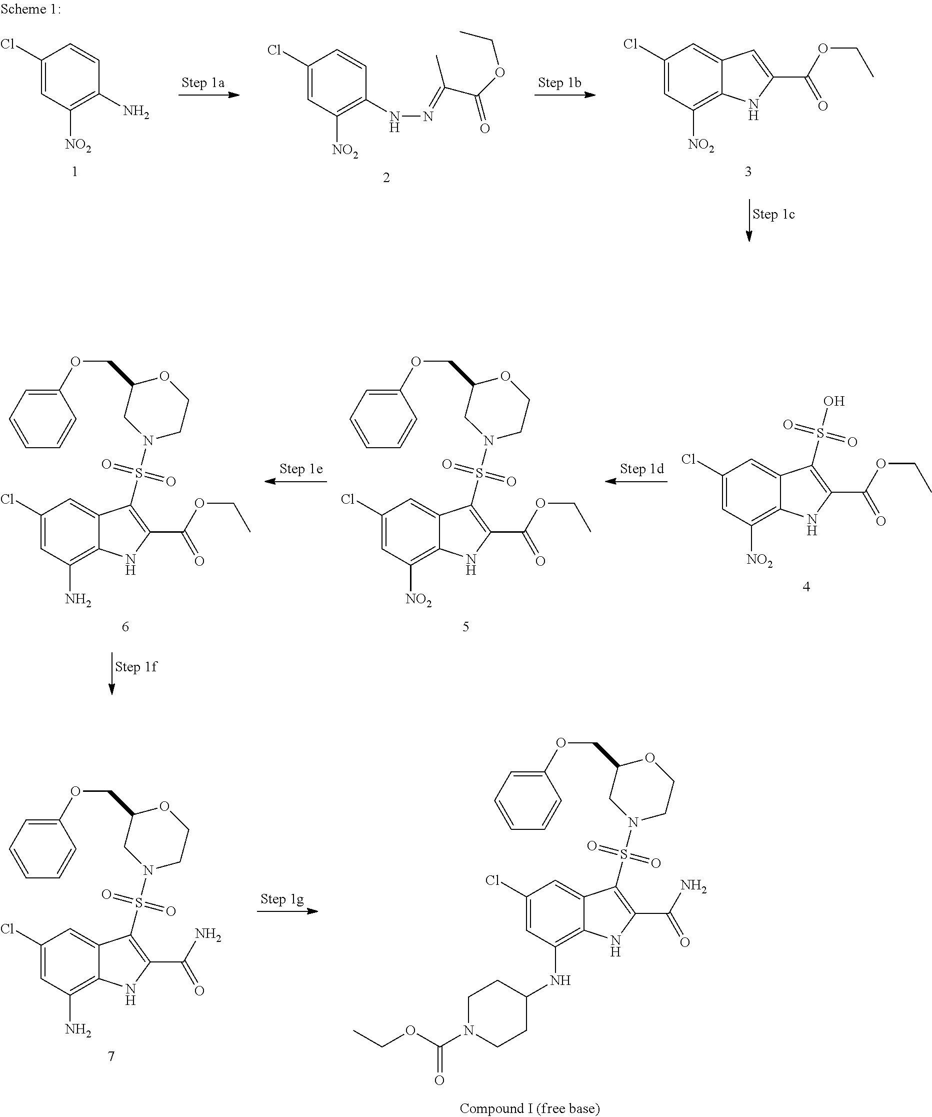

Accordingly, a process for the preparation of the free base of compound I comprises the following steps:

### Step 1a:

Diazotising compound 1 (which is commercially available or may be prepared by methods, well-known in the art):

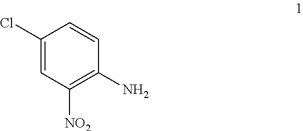 [SMILES: Nc1ccc(Cl)cc1[N+](=O)[O-]]

by reacting it with NaNO₂ and HCl at a temperature range of −10 to 5° C., followed by a dropwise addition of the diazotized mixture to an alkaline solution of the reagent, ethyl 2-methyl-3-oxobutanoate in a base selected from NaOEt, KOH or NaOH in a solvent selected from methanol or ethanol at a temperature range of −20° C. to −15° C. to obtain compound 2.

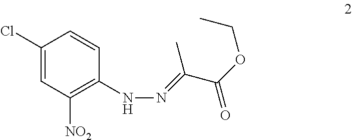 [SMILES: CCOC(=O)/C(C)=N/Nc1ccc(Cl)cc1[N+](=O)[O-]]

In an embodiment, the step 1a is carried out using NaOEt as the base in ethanol as the solvent.

Step 1b:

Cyclising compound 2 obtained in step 1a by reaction with a Lewis acid such as ZnCl₂, AlCl₃, BF₃, P₂O₅ or polyphosphoric acid at a temperature range of 80-120° C. for 5-12 h to obtain compound 3.

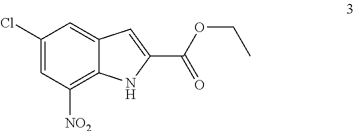 [SMILES: CCOC(=O)c1cc2cc(Cl)cc([N+](=O)[O-])c2[nH]1]

In an embodiment, cyclization of the compound 2 is carried out using polyphosphoric acid in o-phosphoric acid as the Lewis acid at a temperature range of 80-85° C. for 2-3 h.

### Step 1c:

Sulphonating compound 3 obtained in step 1b by reaction with sulphuric acid and acetic anhydride at a temperature range of 0-30° C. for 10-20 h to obtain compound 4.

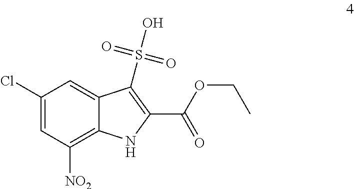 [SMILES: CCOC(=O)c1[nH]c2c([N+](=O)[O-])cc(Cl)cc2c1S(=O)(=O)O]

### Step 1d:

Reacting compound 4 as obtained in step 1c with oxalyl chloride or thionyl chloride in the presence of an organic base selected from triethylamine or pyridine in a solvent selected from DMF, methylene dichloride or a mixture thereof at a temperature range of 25-50° C. for 2-4 h to obtain the corresponding sulphonyl chloride 4A:

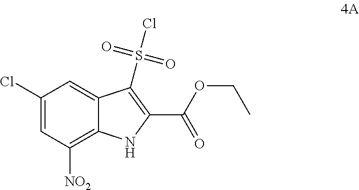 [SMILES: CCOC(=O)c1[nH]c2c([N+](=O)[O-])cc(Cl)cc2c1S(=O)(=O)Cl]

which is optionally isolated; and is then reacted with compound E:

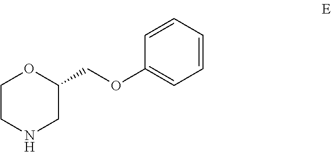 [SMILES: c1ccc(OC[C@@H]2CNCCO2)cc1]

in the presence of an organic base selected from pyridine or triethylamine in a solvent selected from dichloromethane or chloroform at room temperature (25-30° C.) for 1-4 h to obtain compound 5.

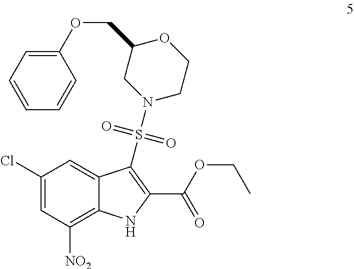 [SMILES: CCOC(=O)c1[nH]c2c([N+](=O)[O-])cc(Cl)cc2c1S(=O)(=O)N1CCO[C@H](COc2ccccc2)C1]

In an embodiment, in the step 1d, compound 4A is isolated prior to reaction with the reagent E.

In another embodiment, the crude compound 5 obtained in step 1d is purified with an alcohol selected from methanol, ethanol, n-propanol, isopropanol or n-butanol to obtain substantially pure compound 5.

In yet another embodiment, the crude compound 5 obtained in step 1d is purified with methanol.

### Step 1e:

Reducing compound 5 obtained in step 1d by reacting it with a reducing agent selected from Fe and NH₄Cl, Zn and HCl or SnCl₂, for 2-8 h in a solvent selected from methanol, ethanol, THF, water or a mixture thereof, to obtain compound 6.

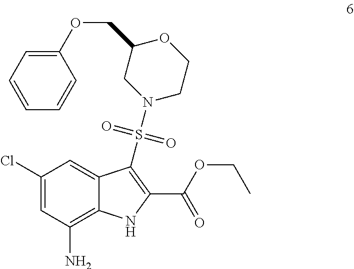 [SMILES: CCOC(=O)c1[nH]c2c(N)cc(Cl)cc2c1S(=O)(=O)N1CCO[C@H](COc2ccccc2)C1]

In an embodiment, in step 1e reduction of compound 5 is carried out using Fe and NH₄Cl as the reducing agent in a mixture of THF, water and ethanol as solvent at a temperature range of 70-80° C. for 2-4 h.

In an embodiment, the residual iron and iron oxides obtained during reduction using Fe and NH₄Cl as reducing agent were removed by using EDTA and chloroform.

In another embodiment, the residual iron and iron oxides obtained during reduction using Fe and NH₄Cl as reducing agent were removed by filtration.

In an embodiment, the crude compound 6 obtained is purified with an alcohol selected from methanol, ethanol, n-propanol, isopropanol or n-butanol to obtain substantially pure compound 6.

In another embodiment, the crude compound 6 obtained is purified with isopropanol.

### Step 1f:

Reacting compound 6 obtained in step 1e with isopropyl alcohol and ammonia at a temperature range of 80-120° C. at a pressure of 0.5-10 kg/cm² for 10-18 h in an autoclave or in a microwave for 10-15 min to obtain compound 7:

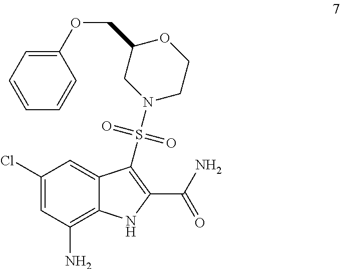 [SMILES: NC(=O)c1[nH]c2c(N)cc(Cl)cc2c1S(=O)(=O)N1CCO[C@H](COc2ccccc2)C1]

In an embodiment, the crude compound 7 is purified with an alcohol selected from methanol, ethanol, n-propanol, isopropanol or n-butanol to obtain substantially pure compound 7.

In an embodiment, the crude compound 7 is purified with isopropanol.

### Step 1g:

Reacting compound 7 obtained in step 1f with compound F:

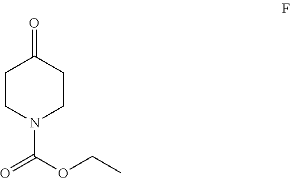

in the presence of trifluoroacetic acid in a base such as sodium triacetoxy borohydride in a solvent selected from dichloromethane or ethyl acetate at room temperature for 0.5-2 h to obtain Compound I as a free base.

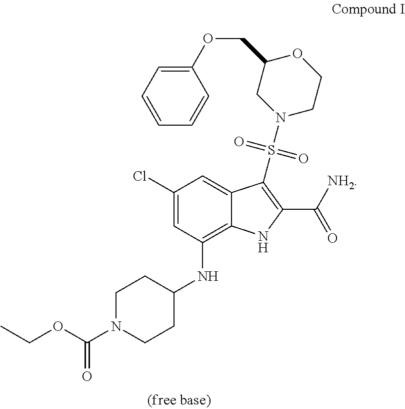 [SMILES: CCOC(=O)N1CCC(Nc2cc(Cl)cc3c(S(=O)(=O)N4CCO[C@H](COc5ccccc5)C4)c(C(N)=O)[nH]c23)CC1]

Process for the preparation of the compound E used in step 1d above employs reaction steps depicted in the following scheme 2:

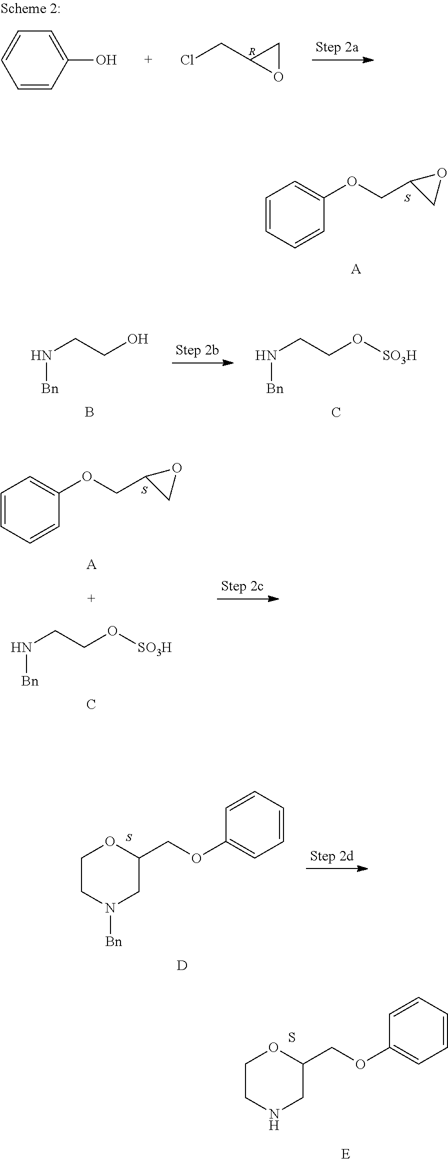

Accordingly, a process for the preparation of Compound E used in step 1d above, comprises the following steps:

### Step 2a:

Reacting commercially available phenol with (R)-2-(chloromethyl)oxirane in the presence of a base selected from aqueous NaOH or aqueous KOH and a phase transfer catalyst such as tetrabutylammonium hydrogen sulphate at a temperature range of 80-120° C. for 1-4 h to obtain Compound A;

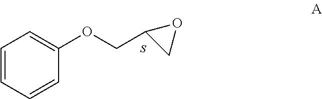

### Step 2b:

### Reacting Compound B;

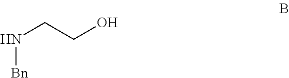 [SMILES: OCCNCc1ccccc1]

with chlorosulfonic acid in a solvent selected from chloroform, carbon tetrachloride, or dichloromethane at 0-10° C. during addition of the acid over a period of 15-30 min, followed by at room temperature for 10-16 h to obtain Compound C;

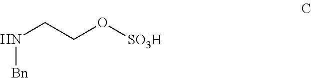 [SMILES: O=S(=O)(O)OCCNCc1ccccc1]

### Step 2c:

Reacting the compound A obtained in step 2a with the compound C obtained in step 2b in the presence of an aqueous base such as NaOH or aqueous KOH in a solvent selected from toluene, dioxane or THF in the presence of a phase transfer catalyst such as tetrabutylammoniun hydrogen sulfate at a temperature range of 30-50° C. for 10-16 h to obtain Compound D;

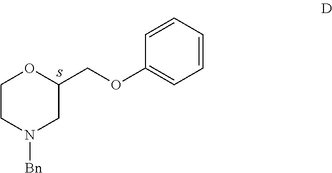

### Step 2d:

Carrying out debenzylation of the compound D by refluxing the said compound D with ammonium formate and 10% Pd/C in an atmosphere of carbon dioxide in a solvent selected from ethanol or methanol at 50-70° C. for 1-3 h to obtain the compound E.

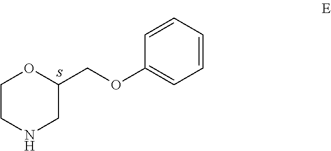

### Utility

In one aspect, this present invention relates to a method of modulating the catalytic activity of PKs (protein kinases) in a subject in need thereof comprising contacting the PK with the crystalline form of compound I. As used herein, the term “modulation” or “modulating” refers to the alteration of the catalytic activity of receptor tyrosine kinases (RTKs), cellular tyrosine kinases (CTKs) and serine-threonine kinases (STKs). In particular, modulating refers to the activation of the catalytic activity of RTKs, CTKs and STKs, preferably the activation or inhibition of the catalytic activity of RTKs, CTKs and STKs, depending on the concentration of the compound or salt to which the RTKs, CTKs or STKs is exposed or, more preferably, the inhibition of the catalytic activity of RTKs, CTKs and STKs.

The term “catalytic activity” as used herein refers to the rate of phosphorylation of tyrosine under the influence, direct or indirect, of RTKs and/or CTKs or the phosphorylation of serine and threonine under the influence, direct or indirect, of STKs.

The term “contacting” as used herein refers to bringing the crystalline form of compound 1 and a target PK together in such a manner that the compound can affect the catalytic activity of the PK, either directly; i.e., by interacting with the kinase itself, or indirectly; i.e., by interacting with another molecule on which the catalytic activity of the kinase is dependent. Such “contacting” can be accomplished “in vitro,” i.e., in a test tube, a petri dish or the like. In a test tube, contacting may involve only a compound and a PK of interest or it may involve whole cells. Cells may also be maintained or grown in cell culture dishes and contacted with the compound in that environment. In this context, the ability of the compound to affect a PK related disorder; i.e., the IC₅₀ of the compound, defined below, can be determined before use of the compound in vivo with more complex living organisms is attempted. For cells outside the organism, multiple methods exist, and are well known to those skilled in the art, to get the PKs in contact with the compound including, but not limited to, direct cell microinjection and numerous transmembrane carrier techniques.

The above-referenced PK is selected from the group comprising an RTK, a CTK or an STK in another aspect of this invention. Preferably, the PK is an RTK.

Furthermore, it is an aspect of this invention that the receptor tyrosine kinase (RTK) whose catalytic activity is modulated by the crystalline form of compound I is selected from the group comprising EGF, HER2, HER3, HER4, IR, IGF-1R, IRR, PDGFRα, PDGFRβ, TrkA, TrkB, TrkC, HGF, CSFIR, C-Kit, C-fms, Flk-1R, Flk4, KDR/Flk-1, Flt-1, FGFR-1R, FGFR-1R, FGFR-3R and FGFR-4R. Preferably, the receptor protein kinase is selected from IR, IGF-1R, or IRR.

In addition, it is an aspect of this invention that the cellular tyrosine kinase whose catalytic activity is modulated by the crystalline form of compound I is selected from the group consisting of Src, Frk, Btk, Csk, Abl, ZAP70, Fes, Fps, Fak, Jak, Ack, Yes, Fyn, Lyn, Lck, Blk, Hck, Fgr and Yrk.

Another aspect of this invention is that the serine-threonine protein kinase whose catalytic activity is modulated by the crystalline form of compound I is selected from the group consisting of CDK2 and Raf.

In another aspect, this invention relates to a method for treating or preventing a PK-related disorder in a subject in need of such treatment comprising administering to the subject a therapeutically effective amount of the crystalline form of compound I.

The term “subject” as used herein refers to an animal, preferably a mammal, and most preferably a human.

The term “mammal” used herein refers to warm-blooded vertebrate animals of the class Mammalia, including humans, characterized by a covering of hair on the skin and, in the female, milk-producing mammary glands for nourishing the young. The term mammal includes animals such as cat, dog, rabbit, bear, fox, wolf, monkey, deer, mouse, pig as well as human.

As used herein, “PK-related disorder,” “PK driven disorder,” and “abnormal PK activity” all refer to a condition characterized by inappropriate (i.e., diminished or, more commonly, excessive) PK catalytic activity, where the particular PK can be an RTK, a CTK or an STK. Inappropriate catalytic activity can arise as the result of either: (1) PK expression in cells which normally do not express PKs; (2) increased PK expression leading to unwanted cell proliferation, differentiation and/or growth; or, (3) decreased PK expression leading to unwanted reductions in cell proliferation, differentiation and/or growth. Excessive-activity of a PK refers to either amplification of the gene encoding a particular PK or its ligand, or production of a level of PK activity which can correlate with a cell proliferation, differentiation and/or growth disorder (that is, as the level of the PK increases, the severity of one or more symptoms of a cellular disorder increase as the level of the PK activity decreases).

“Treat,” “treating” or “treatment” with regard to a PK-related disorder refers to alleviating or abrogating the cause and/or the effects of a PK-related disorder.

As used herein, the terms “prevent”, “preventing” and “prevention” refer to a method for barring a mammal from acquiring a PK-related disorder in the first place.

The term “administration” and variants thereof (e.g., “administering” a compound) in reference to the crystalline form of compound I means introducing the compound into the system of the animal in need of treatment. When the crystalline form of compound I is provided in combination with one or more other therapeutically active agents (e.g., a cytotoxic agent, etc.), “administration” and its variants are each understood to include concurrent and sequential introduction of the compound or prodrug thereof and other agents.

The term “therapeutically effective amount” as used herein means that amount of active compound or pharmaceutical agent (i.e. the crystalline form of Compound I) that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

The protein kinase-related disorder may be selected from the group comprising an RTK, a CTK or an STK-related disorder in a further aspect of this invention. Preferably, the protein kinase-related disorder is an RTK-related disorder.

In yet another aspect of this invention, the above referenced PK-related disorder may be selected from the group consisting of an EGFR-related disorder, a PDGFR-related disorder, an IGFR-related disorder and a flk-related disorder.

The above referenced PK-related disorder may be a cancer selected from, but not limited to astrocytoma, basal or squamous cell carcinoma, brain cancer, gliobastoma, bladder cancer, breast cancer, colon carcinoma, colorectal cancer, chrondrosarcoma, cervical cancer, adrenal cancer, choriocarcinoma, esophageal cancer, endometrial carcinoma, erythroleukemia, Ewing's sarcoma, gastrointestinal cancer, head and neck cancer, hepatoma, glioma, hepatocellular carcinoma, leukemia, leiomyona, melanoma, non-small cell lung cancer, neural cancer, ovarian cancer, pancreatic cancer, prostate cancer, renal cell carcinoma, rhabdomyosarcoma, small cell lung cancer, thymona, thyroid cancer, testicular cancer or osteosarcoma in a further aspect of this invention.

More preferably, the PK-related disorder is a cancer selected from breast cancer, colon carcinoma, colorectal cancer, Ewing's sarcoma or rhabdosarcoma.

The present invention therefore relates to a crystalline form of Compound I for use in the treatment of diseases or disorders mediated by Insulin-Like-Growth Factor I Receptors (IGF-IR) or Insulin Receptors (IR) comprising administering to a subject in need thereof, a therapeutically effective amount of the crystalline form of Compound I.

In an embodiment, the present invention relates to the crystalline form of Compound I for use in the treatment of diseases or disorders mediated by Insulin-Like-Growth Factor I Receptors or Insulin Receptors, wherein the Insulin-Like-Growth Factor I Receptor and Insulin Receptor mediated disease or disorder is cancer.

Accordingly, in an embodiment, the present invention relates to the crystalline form of Compound I for use in the treatment of cancer.

The present invention also encompasses a method of treating or preventing cancer in a mammal in need of such treatment which comprises administering to said mammal a therapeutically effective amount of the crystalline form of the compound I.

In one embodiment, the present invention relates to a use of the crystalline form of Compound I for the manufacture of a medicament for the treatment of diseases or disorders mediated by Insulin-Like-Growth Factor I Receptor (IGF-IR) and Insulin Receptor (IR).

In another embodiment, the present invention relates to the use of the crystalline form of Compound I for the manufacture of a medicament for the treatment of diseases or disorders mediated by Insulin-Like-Growth Factor I Receptor and Insulin Receptor, wherein the Insulin-Like-Growth Factor I Receptor and Insulin Receptor mediated disease or disorder is cancer.

Accordingly, in an embodiment, the present invention relates to the use of the crystalline form of Compound I for the manufacture of a medicament for the treatment of cancer.

Types of cancers which may be treated using the crystalline form of the compound I include, but are not limited to astrocytoma, basal or squamous cell carcinoma, brain cancer, gliobastoma, bladder cancer, breast cancer, colon carcinoma, colorectal cancer, chrondrosarcoma, cervical cancer, adrenal cancer, choriocarcinoma, esophageal cancer, endometrial carcinoma, erythroleukemia, Ewing's sarcoma, gastrointestinal cancer, head and neck cancer, hepatoma, glioma, hepatocellular carcinoma, leukemia, leiomyona, melanoma, non-small cell lung cancer, neural cancer, ovarian cancer, pancreatic cancer, prostate cancer, renal cell carcinoma, rhabdomyosarcoma, small cell lung cancer, thymona, thyroid cancer, testicular cancer or osteosarcoma. Preferably, the cancer being treated is selected from breast cancer, colon carcinoma, colorectal cancer, Ewing's sarcoma or rhabdosarcoma.

The above-referenced PK-related disorder may be an IGFR-related disorder selected from diabetes, an autoimmune disorder, Alzheimer's and other cognitive disorders, a hyperproliferation disorder, aging, cancer, acromegaly, Crohn's disease, endometriosis, diabetic retinopathy, restenosis, fibrosis, psoriasis, osteoarthritis, rheumatoid arthritis, an inflammatory disorder and angiogenesis in yet another aspect of this invention.

A method of treating or preventing retinal vascularization which is comprised of administering to a mammal in need of such treatment a therapeutically effective amount of the crystalline form of the compound I is also encompassed by the present invention. Methods of treating or preventing ocular diseases, such as diabetic retinopathy and age-related macular degeneration, are also part of the invention.

Also included within the scope of the present invention is a method of treating or preventing inflammatory diseases, such as rheumatoid arthritis, psoriasis, contact dermatitis and delayed hypersensitivity reactions, as well as treatment or prevention of bone associated pathologies selected from osteosarcoma, osteoarthritis, and rickets.

Other disorders which might be treated with the compound of this invention include, without limitation, immunological and cardiovascular disorders such as atherosclerosis.

Also included in the scope of the claims is a method of treating cancer that comprises administering a therapeutically effective amount of the crystalline form of compound I in combination with radiation therapy and/or in combination with a second compound which is a therapeutically effective compound selected from: an estrogen receptor modulator, an androgen receptor modulator, a retinoid receptor modulator, a cytotoxiccytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase inhibitors, agents that interfere with receptor tyrosine kinases (RTKs) and an agent that interferes with a cell cycle checkpoint.

The instant invention also includes a pharmaceutical composition useful for treating or preventing cancer that comprises a therapeutically effective amount of the crystalline form of the compound I along with said second compound.

The PKs whose catalytic activity is modulated by the compound of this invention include protein tyrosine kinases of which there are two types, receptor tyrosine kinases (RTKs) and cellular tyrosine kinases (CTKs), and serine-threonine kinases (STKs). RTK-mediated signal transduction, is initiated by extracellular interaction with a specific growth factor (ligand), followed by receptor dimerization (or conformational changes in the case of IR, IGF-1R or IRR), transient stimulation of the intrinsic protein tyrosine kinase activity, autophosphorylation and subsequent phosphorylation of other substrate proteins.

In another aspect, the protein kinase (PK), the catalytic activity of which is modulated by contact with the crystalline form of the compound I, is a protein tyrosine kinase (PTK), more particularly, a receptor protein tyrosine kinase (RTK). Among the RTKs whose catalytic activity can be modulated with the compound of this invention, or salt thereof, are, without limitation, EGF, HER2, HER3, HERO, IR, IGF-1R, IRR, PDGFRα, PDGFRβ, TrkA, TrkB, TrkC, HGF, CSFIR, C-Kit, C-fms, Flk-1R, Flk4, KDR/Flk-1, Flt-1, FGFR-1R, FGFR-2R, FGFR-3R and FGFR-4R. Preferably, the RTK is selected from IGF-1R.

The protein tyrosine kinase whose catalytic activity is modulated by contact with the crystalline form of the compound I, can also be a non-receptor or cellular protein tyrosine kinase (CTK). Thus, the catalytic activity of CTKs such as, without limitation, Src, Frk, Btk, Csk, Abl, ZAP70, Fes, Fps, Fak, Jak, Ack, Yes, Fyn, Lyn, Lck, Blk, Hck, Fgr and Yrk, may be modulated by contact with the crystalline form of the compound I.

Still another group of PKs which may have their catalytic activity modulated by contact with crystalline form of the compound I are the serine-threonine protein kinases such as, without limitation, CDK2 and Raf.

The present invention is directed to crystalline form of the compound I which modulates RTK, CTK and/or STK mediated signal transduction pathways as a therapeutic approach to cure many kinds of solid tumors, including, but not limited to, carcinomas, sarcomas including Kaposi's sarcoma, erythroblastoma, glioblastoma, meningioma, astrocytoma, melonoma and myoblastoma. Treatment or prevention of non-solid tumor cancers such as leukemia are also contemplated by this invention. Indications may include, but are not limited to brain cancers, bladder cancers, ovarian cancers, gastric cancers, pancreatic cancers, colon cancers, blood cancers, breast cancers, prostrate cancers, renal cell carcinomas, lung cancer and bone cancers.

Further examples, without limitation, of the types of disorders related to inappropriate PK activity that the compound described herein may be useful in preventing, treating and studying, are cell proliferative disorders, fibrotic disorders and metabolic disorders.

These and other aspects of the invention will be apparent from the teachings contained herein.

### Compositions and Formulations

Pharmaceutical compositions of the compound of the present invention are a further aspect of this invention.

As used herein, the term “composition” is intended to encompass a product comprising the specified ingredients in the specified amounts, as well as any product which results, directly or indirectly, from combination of the specified ingredients in the specified amounts.

As used herein, the term “active ingredient” refers to any substance or mixture of substances intended to be used in the manufacture of a drug (medicinal) product, which substance(s) are intended to furnish pharmacological activity or other direct effect in the diagnosis, cure, mitigation, treatment, or prevention of disease or to affect the structure or function of the body. Reference: Manufacturing, Processing, or Holding Active Pharmaceutical Ingredients FDA Guidance. In the context of the present invention, the term “active ingredient” refers to Compound I.

The present invention also encompasses a pharmaceutical composition useful in the treatment of cancer, comprising the administration of a therapeutically effective amount of the crystalline form of the compound I, with or without pharmaceutically acceptable carriers or diluents. Suitable compositions of this invention include aqueous solutions comprising the crystalline form of the compound I and pharmacologically acceptable carriers, e.g., saline, at a pH level, e.g., 7.4. The solutions may be introduced into a patient's bloodstream by local bolus injection.

The crystalline form of the compound I may be administered to mammals, preferably humans, either alone or, preferably, in combination with pharmaceutically acceptable carriers, excipients or diluents, optionally with known adjuvants, such as alum, in a pharmaceutical composition, according to standard pharmaceutical practice. The crystalline form of the compound I can be administered orally or parenterally, including the intravenous, intramuscular, intraperitoneal, subcutaneous, rectal and/or topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropyl-cellulose, or a time delay material such as ethyl cellulose, cellulose acetate buryrate may be employed.

Formulations for oral use may be presented as hard gelatin capsules wherein the active ingredient is mixed with an inert solid diluent, for example, calcium carbonate, calcium phosphate, kaolin, lactose or dried cornstarch, or as soft gelatin capsules wherein the active ingredient is mixed with water soluble carrier such as polyethyleneglycol or an oil medium, for example peanut oil, liquid paraffin, or olive oil. For oral use of the compound according to this invention, particularly for chemotherapy, the compound may be administered, for example, in the form of a tablet or a capsule, or as an aqueous solution or suspension. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form ofan oil-in-water emulsions. The oily phase may be a vegetable oil, for example olive oil or arachis oil, or a mineral oil, for example liquid paraffin phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solution. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, for example as a solution in 1,3-butane diol. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compound of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of the present invention are employed. (For purposes of this application, topical application shall include mouth washes and gargles.)

The compound of the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. The compound of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When the compound of the present invention is administered into a human subject, the daily dosage will normally be determined by the prescribing physician with the dosage generally varying according to the age, weight, and response of the individual patient, as well as the severity of the patient's symptoms.

In one exemplary application, a suitable amount of the compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

The use of all of these approaches in combination with the instant compound described herein are within the scope of the present invention.

In respect of the schemes of preparation (Scheme(s) 1 and 2) depicted herein above, it may be noted that in addition to other standard manipulations that are known in the literature or exemplified in the experimental procedures. Substituent numbering, as shown in the schemes, does not necessarily correlate to that used in the claims. It should also be noted that any carbon as well as heteroatom with unsatisfied valences in the text, schemes, examples and Tables herein is assumed to have the sufficient number of hydrogen atom(s) to satisfy the valences. According to the present disclosure, the definition “substantially pure compound” means compound comprising less than 10%, preferably less than 5% of undesired chemical impurities, which can be determined, for example, by HPLC.

Abbreviations, which may be used in the description of the chemistry and in the Examples that follow, include:
ATCC American Type Culture Collection, USA;
ATP Adenosine triphosphate;
Ac₂O Acetic anhydride;
AcOH Acetic acid;
AlCl₃ Aluminium chloride;
BF₃ Boron trifluoride;
CDCl₃ Deuterated chloroform;
CO₂ Carbon dioxide;
DCM Dichloromethane;
DMF N,N-Dimethylformamide;
DMSO Dimethyl sulfoxide;
DMSO-d₆ Deuterated dimethyl sulfoxide;
D₂O Deuterated water;
EDTA Ethylenediaminetetraacetic acid;
Et₃N Triethylamine;
EtOAc Ethyl acetate;
EtOH Ethanol;
FBS Fetal bovine serum (Gibco, USA);
Fe Iron;
GC Gas chromatography;
HPLC High-performance liquid chromatography;
HCl Hydrochloric acid;
LOD Loss on drying;
KOH Potassium hydroxide;
M.C. Moisture content;
MeOH Methanol;
MS Mass Spectroscopy;
NMT Not more than;
NLT Not less than;
NH₃ Ammonia
NaOH Sodium hydroxide;
NaOEt Sodium ethoxide;
Na₂SO₄ Sodium sulfate;
NaNO₂ Sodium nitrite;
NH₄Cl Ammonium chloride;
NMR Nuclear Magnetic Resonance;
PBS Phosphate buffered saline (Sigma Aldrich, USA);
Pd/C Palladium over activated charcoal or Palladium-carbon;
P₂O₅ Phosphorous pentoxide;
SnCl₂ Stannous chloride;
RT Room Temperature;
TFA Trifluoroacetic acid;
THF Tetrahydrofuran;
Zn Zinc;
ZnCl₂ Zinc chloride;

### Cell-Lines (Source: ATCC, USA):

Colo205: Human colon adenocarcinoma cell-line
MCF-7: Human breast adenocarcinoma cell-line
A673: Ewing tumor cell-line
SK-ES: Human Ewing's sarcoma cell-line
RD-ES: Human Ewing's sarcoma cell-line
HCT-116: Colon cancer cell-line
RD: Rhabdomyosarcoma cell-line

### EXAMPLES

### Example 1

### Ethyl 2-(2-(4-chloro-2-nitrophenyl)hydrazono)propanoate (Compound 2)

To an ice-cold solution of NaOEt (Spectrochem, 49.3 g, 0.724 mol) in ethanol (Commercial grade, 500 mL) at −15° C. to −10° C., was added ethyl-2-methyl acetoacetate (Aldrich, 50.95 g, 0.353 mol) drop-wise maintaining temperature below −15° C. over a period of 0.45-1 h and the resulting mixture was stirred for 45 min at −15° C. to −20° C. Simultaneously a cold diazonium salt solution was prepared by addition of sodium nitrite (Spectrochem, 27.9 g, 0.405 mol) to a solution of 2-nitro-4-chloro aniline (Aldrich, 50 g, 0.289 mol) in a mixture of conc. HCl (100 mL) and water (225 mL) at −10° C. to −5° C. The diazonium salt mixture was then added into the ethanol solution of ethyl-2-methyl acetoacetate with constant stirring, maintaining the temperature below −10° C. The reaction was stirred for another 30 to 40 min (pH=2-3). The solid was then filtered by suction filtration to yield crude compound 2, which is washed with water (150 mL) and again filtered by suction filtration. The compound is dried at 12-16 h at 45-50° C. to afford pure compound 2.

Yield range: 66-72%; HPLC Purity: ˜65%; Moisture content: NMT 1%; ¹H NMR (300 MHz, DMSO-d₆): δ 10.87 (s, 1H), 8.19 (s, 1H), 8.01-7.99 (d, J=8.4 Hz, 1H), 7.57-7.54 (d, J=7.8 Hz, 1H), 4.37-4.35 (q, 2H), 2.24 (s, 3H), 1.40 (t, 3H); MS: m/z 284 (M−H)⁻.

### Example 2

### Ethyl 5-chloro-7-nitro-1H-indole-2-carboxylate (Compound 3)

A mixture of o-phosphoric acid (Spectrochem, 38.4 mL), polyphosphoric acid (PPA) (Spectrochem, 384 g) and the compound 2 of example 1 (128 g, 0.4481 mol) were heated with stirring at 80-85° C. for 2-3 h. On completion of the reaction, the temperature was reduced to 55-60° C. and water was added to the reaction mixture slowly with stirred for another 30 minutes. The solid precipitate was filtered and redissolved in EtOAc, treated with charcoal (Norit CA1, 6.4 g) for 1 h and filtered through a celite bed. The organic layer was washed using 5% sodium bicarbonate (Merck, 2.56 L) and 10% NaCl solution (1.2 L), dried over anhydrous Na₂SO₄ (Merck, 100 g) and evaporated to yield the crude compound 3, which was treated with n-heptane (768 mL) and filtered to afford the title compound 3, which was dried at −50° C. for 15-16 h.

Yield range: 32-42%, HPLC Purity: ˜85%, LOD: NMT 2% w/w, ¹H NMR (300 MHz, DMSO-d₆): δ 10.31 (s, 1H), 8.27-8.26 (d, J=1.5 Hz, 1H), 8.01-8.01 (d, J=1.2 Hz, 1H), 7.30-7.27 (s, 1H), 4.51-4.44 (q, 2H), 1.48-1.41 (t, 3H); MS: m/z 267 (M−H)⁻.

### Example 3

### 5-Chloro-2-(ethoxycarbonyl)-7-nitro-1H-indole-3-sulfonic acid (Compound 4)

To compound 3 of example 2 (84 g, 0.3128 mol) was added acetic anhydride (S.D. Finechem, 336 mL) at room temperature. The reaction mixture was subsequently cooled to 0-10° C., and sulphuric acid (Rankem, 153.3 g, 83.31 mL) was added drop wise over a period of 35-40 min. The reaction was stirred for 15-16 h at room temperature to ensure completion of the reaction. The solid was then filtered by suction filtration to obtain crude compound 3, which was washed with AcOH (Spectrochem, 84 mL), subsequently washed with EtOAc (Commercial grade, 84 mL) and dried at 45-50° C. to afford the title compound 4.

Yield range: 54-62%; HPLC Purity: ˜90%; LOD: NMT 5% w/w, M. C.: NMT 5% w/w; ¹H NMR (300 MHz, DMSO-d6) δ 12.28 (s, 1H), s 8.357-8.351 (d, J=1.8 Hz, 1H), 8.18-8.17 (d, J=1.8 Hz, 1H), 4.33-4.25 (q, 2H), 1.33-1.29 (t, 3H); MS: m/z 347 (M−H)⁻.

### Example 4

### Ethyl 5-chloro-3-(chlorosulfonyl)-7-nitro-1H-indole-2-carboxylate (Compound 4A)

Compound 4 of example 3 (40 g, 0.115 mol) was suspended in dichloromethane (Commercial grade, 550 mL) to which catalytic amount of DMF (A.R. Grade ˜20 mL) was added and the mixture was stirred at room temperature till a clear solution was obtained. Oxalyl chloride (Spectrochem, 72.9 g, 50 mL, 0.574 mol) in dichloromethane (250 mL) was added to the reaction mixture drop wise over a period of 30-45 min at room temperature. The reaction mixture was heated at 40° C. with stirring for 2-3 h. After evaporating a portion of dichloromethane (3-5 vol), the reaction mixture is cooled to 0-5° C. and maintained under an atmosphere of nitrogen for 1.5-2 h. The crude compound obtained was filtered, washed with chilled dichloromethane (40 mL) and dried at 40-45° C. to afford the title compound 4A.

Yield Range: 76-85%; HPLC: 95%; LOD: NMT 3% w/w.

### Example 5

### (S)-Ethyl 5-chloro-7-nitro-3-(2-(phenoxymethyl)morpholinosulfonyl)-1H-indole-2-carboxylate (Compound 5)

To a solution of (S)-2-(phenoxymethyl)morpholine (Compound E, 17.3 g, 0.09 mol) in dichloromethane (Commercial grade, 300 mL), triethylamine (L.R. grade, 16.5 g, 0.163 mol) was added over a period of 30-45 min at −5 to −10° C. At this temperature, compound 4a of example 4 (30 g, 0.081 mol) was added in portions over a period of 1 h. The reaction was then stirred for 2-3 h at 30° C. On completion of the reaction, the reaction was quenched with water (250 mL). The organic layer was dried over anhydrous sodium sulfate (10 g). The organic layer was concentrated to a small volume and the residue was treated with methanol (Commercial grade, 360 mL) at room temperature for 30-60 min. The solid obtained is suction-filtered and washed with chilled methanol (60 mL). The solid is dried at 45-50° C. to afford the title compound 5, which was purified using methanol.

Yield range: 63-80%; HPLC Purity: NLT 97%; Chiral Purity: NLT 95% ee; LOD: NMT 2.0% w/w; ¹H NMR (300 MHz, DMSO-d₆): δ 13.46 (s, 1H), 8.338-8.332 (d, J=1.8 Hz 1H), 8.26-8.25 (d, J=1.8 Hz, 1H), 7.29-7.24 (m, 2H), 6.95-6.88 (m, 3H), 4.41-4.34 (q, 2H), 3.98-3.93 (m, 3H), 3.81-3.77 (m, 2H), 3.67-3.58 (m, 2H), 2.60-2.49 (m, 2H), 1.32-1.28 (t, 3H); MS: m/z 524 (M+H)⁺.

### Example 6

### (S)-Ethyl 7-amino-5-chloro-3-(2-(phenoxymethyl)morpholinosulfonyl)-1H-indole-2-carboxylate (Compound 6)

Compound 5 of example 5 (27 g, 0.0516 mol), iron powder (Merck, 100 mesh electrolytic, 9.25 g, 0.1652 mol), ammonium chloride (Merck, 8.83 g, 0.1652 mol) was added to a mixture of ethanol (Commercial grade, 151 mL), THF (Spectrochem, 75 mL) and water (37 mL). The reaction mixture was heated to 70-80° C. and maintained at that temperature for 3-4 h. On completion of the reaction, the reaction mixture was cooled to 55-60° C. and filtered hot through a bed of celite. The celite bed was further washed with EtOAc (Commercial grade, 135 mL). The filtrate was concentrated to reduce the volume, which was chased with EtOH (Commercial grade, 54 mL), water (540 mL) was added and stirred at room temperature for 30-45 min. The solid obtained was suction-filtered, washed with water (54 mL) and dried at 45-50° C. for 12-16 h to afford the title compound 6. The compound 6 obtained may be optionally purified further by treatment with isopropyl alcohol (Commercial grade, 130 mL) followed by filtration and drying.

Yield range: 63-72%; HPLC Purity: NLT 95%; LOD: NMT 3% w/w; ¹H NMR (300 MHz, DMSO-d₆): δ 12.66 (s, 1H), 7.29-7.24 (m, 2H), 7.17 (s, 1H), 6.95-6.88 (m, 3H), 6.52 (s, 1H), 6.00 (bs, 2H), 4.41-4.34 (q, 2H), 3.99-3.90 (m, 3H), 3.81-3.78 (m, 2H), 3.61-3.52 (m, 2H), 2.59-2.50 (m, 2H), 1.34-1.22 (t, 3H); MS: m/z 494.1 (M+H)⁺.

### Example 7

### (S)-7-Amino-5-chloro-3-(2-(phenoxymethyl)morpholinosulfonyl)-1H-indole-2-carboxamide (Compound 7)

Compound 6 of example 6 (36 g, 0.0728 mol) was dissolved in isopropyl alcohol (IPA) (Commercial grade, 720 mL) in a 2 L autoclave and the reaction mixture was cooled to −10 to −5° C. for 30 min. Ammonia gas was purged for about 30 minutes at the same temperature and maintaining a pressure of 2 kg/m². The reaction mixture was heated to 105-110° C. for 14-16 h. The reaction was cooled to −10° C. and unloaded from the autoclave. The organic layer was partially distilled below 50° C. and the residue was cooled to room temperature and maintained at room temperature for 45-60 min. The solid was suction-filtered, washed with IPA (2×36 mL) and dried at 45-50° C. to afford the title compound 7, which was purified using isopropyl alcohol.

Yield range: 73-87%, HPLC Purity: NLT 96%, LOD: NMT 3% w/w, ¹H NMR (300 MHz, DMSO-d₆): δ 12.59 (s, 1H), 8.30-8.23 (d, J=21.0 Hz, 2H), 7.28-7.23 (m, 2H), 7.108-7.102 (d, J=1.8 Hz, 1H), 6.94-6.87 (m, 3H), 6.49-6.48 (d, J=1.8 Hz, 1H), 6.01 (bs, 2H), 4.03-3.94 (m, 2H), 3.90-3.79 (m, 2H), 3.68-3.46 (m, 3H), 2.50-2.31 (m, 2H). MS: m/z 465.1 (M+H)⁺.

### Example 8

### (S)-Ethyl 4-((2-carbamoyl-5-chloro-3-((2-(phenoxymethyl)morpholino)sulfonyl)-1H-indol-7-yl)amino)piperidine-1-carboxylate (Compound I as Free Base)

Compound 7 of example 7 (18 g, 0.0387 mol) and ethyl 4-oxopiperidine-1-carboxylate (Oakwood Corporation Inc., 9.96 g, 8.7 mL, 0.0581 mol) were taken in dichloromethane (Commercial grade, 360 mL) and the turbid solution was stirred for 20 h at room temperature. Trifluoroacetic acid (TFA) (Merck, 2.98 mL, 0.0387 mol) in dichloromethane (18 mL) was added dropwise and stirred for 2 h. Following this, sodium tri-acetoxyborohydride (Spectrochem, 24.55 g, 0.1163 mol) was added and the reaction mixture was stirred for another 1.5 h at room temperature. The reaction mass was partially concentrated and the residue was dissolved in ethyl acetate (Commercial grade, 360 mL).

After cooling to room temperature, the organic layer was washed with 5% sodium bicarbonate solution (Merck, 180 mL) and 20% brine (180 mL). The organic phase was filtered through a celite bed, which was washed with ethyl acetate (36 mL) and dried over anhydrous sodium sulphate (Merck, 20 g). The organic solvent was distilled under 45° C. to yield a crude solid, which was treated with dichloromethane (180 mL) at room temperature for 30-40 min, suction-filtered and dried at 45-50° C. for 12-14 h to afford the title compound I as a free base, which was purified using dichloromethane.

Yield range: 67-75%; HPLC Purity: NLT 97%; LOD: NMT 2% w/w; ¹H NMR (300 MHz, DMSO-d₆): δ 12.66 (s, 1H), 8.31-8.31 (d, J=12.6 Hz, 2H), 7.28-7.23 (t, J=8.1 Hz, 2H), 7.14-7.13 (d, J=1.2 Hz, 1H), 6.95-6.87 (m, 2H), 6.474-6.471 (d, J=0.9 Hz, 1H), 6.38-6.36 (d, J=7.2, 1H), 4.08-3.94 (m, 2H), 3.97-3.91 (m, 4H), 3.82-3.80 (m, 2H), 3.67-3.64 (d, J=10.5 Hz, 2H), 3.58-3.43 (m, 2H), 3.07 (m, 2H), 2.45-2.30 (m, 3H), 2.02-1.98 (d, J=9.9 Hz, 2H), 1.37-1.26 (m, 2H), 1.21-1.17 (t, J=6.9 Hz, 3H); MS: m/z 620.2 (M+H)⁺.

### Example 9

### Methanesulfonic acid salt of (S)-ethyl 4-((2-carbamoyl-5-chloro-3-((2-(phenoxymethyl)morpholino)sulfonyl)-1H-indol-7-yl)amino)piperidine-1-carboxylate (Compound I as Mesylate)

To isopropyl acetate (Commercial grade, 200 mL), compound I of example 8 (25 g, 0.0403 mol) was added and stirred at room temperature for 15-30 min. To the clear solution obtained, 10% activated charcoal (Norit (CA1 0155-9), 2.5 g) and 5% SiThiol (Silicycle, 1.25 g) were added and the reaction mixture was stirred for 30-35 min. The compound was then filtered through a celite bed (Commercial grade, 75 g), which was washed with isopropyl acetate (Commercial grade, 25 mL). After complete removal of solvent by distillation, the residue was dissolved in isopropyl acetate (Commercial grade, 200 mL) at room temperature. To this methane sulfonic acid (Avra Labs, 4.27 g, 0.0444 mol) was added over a period of 15-30 min at room temperature. The reaction mixture was heated to 75-80° C. and maintained at that temperature for 4.5-5 h to complete crystallization. The reaction mixture was cooled and maintained at room temperature for 1 h followed by at 0-5° C. for 2 h. The solid obtained as crystals were suction-filtered, washed with chilled isopropyl acetate (25 mL) and dried at 50-55° C. to afford the crystals of the title compound I as mesylate salt.

Yield range: 78-86%; HPLC Purity: NLT 97%; Chiral Purity: NLT 95% ee; LOD: ˜1.0%; ¹H NMR (300 MHz, DMSO-d₆): δ 12.66 (s, 1H), 8.30-8.26 (d, J=13.2 Hz, 2H), 7.28-7.23 (t, J=7.5 Hz, 2H), 7.14 (s, 1H), 6.94-6.87 (m, 3H), 6.47 (s, 1H), 4.06-4.01 (m, 2H), 3.95-3.90 (m, 4H), 3.81 (m, 1H), 3.67-3.59 (m, 2H), 3.50-3.46 (m, 2H), 3.07 (m, 2H), 2.44 (s, 3H), 2.37-2.30 (m, 2H), 2.02-1.98 (d, J=10.5 Hz, 2H), 1.75 (m, 1H), 1.34-1.31 (m, 2H), 1.21-1.17 (t, J=7.2 Hz, 3H); IR (Perkin Elmer, KBr): cm⁻¹ 3400, 1698, 1687, 1338, 1155.

### Characterisation of the Crystalline Form of Compound I:

The crystalline form was characterised using following procedures:

### 1. X-Ray Powder Diffraction (XRPD) Pattern Analysis:

X-Ray diffractograms of the crystalline form of Compound I was recorded on a X-Ray difractometer, Bruker, D8 Advance, LynxEye detector, X-Ray tube with Cu target anode, slit 0.3, antiscatter slit 1°, Power 40 kV, 40 mA, Scanning speed 0.25 sec/step, 0.02 deg, Wave length: 1.5406 A

The X-Ray diffractograms were recorded for the crystalline form of Compound I, obtained using solvent of crystallisation selected from THF, 2-methyl tetrahydrofuran, a mixture of 2-methyl tetrahydrofuran and toluene, a mixture of 2-methyl tetrahydrofuran and heptane, methylethylketone, ethyl acetate or isopropyl acetate and were found to be identical, indicating that an identical crystalline form of Compound I was obtained with each of the above-mentioned solvents.

Table 1 indicates the main peaks of % intensity greater than 10, at an angle of refraction 2-theta of 9.22, 11.92, 13.58, 15.74, 18.37, 18.65, 18.95, 19.37, 19.59, 20.33, 20.92, 22.48, 22.79, 23.97, 24.19, 24.59, 28.48±0.2°, obtained for the crystalline form of Compound I, obtained using isopropyl acetate as crystallization solvent.

TABLE 1

| Angle | d value | Intensity |
|---|---|---|
| 2-Theta ° | Angstrom | % |
| 7.662 | 11.52853 | 7.4 |
| 8.93 | 9.89513 | 3.8 |
| 9.22 | 9.58418 | 45 |
| 11.423 | 7.74044 | 5.1 |
| 11.918 | 7.4213 | 13.5 |
| 12.898 | 6.85822 | 5.6 |
| 13.151 | 6.72656 | 3.5 |
| 13.577 | 6.5169 | 13.6 |
| 14.322 | 6.1793 | 5.2 |
| 15.376 | 5.75792 | 15.5 |
| 15.745 | 5.62406 | 20.7 |
| 16.275 | 5.44207 | 12.5 |
| 17.478 | 5.07012 | 5.4 |
| 17.767 | 4.98804 | 4.8 |
| 18.369 | 4.82593 | 43.3 |
| 18.649 | 4.75422 | 16.8 |
| 18.953 | 4.6787 | 19.9 |
| 19.374 | 4.57784 | 12.9 |
| 19.586 | 4.52887 | 25.1 |
| 19.855 | 4.46806 | 5.7 |
| 20.331 | 4.36445 | 34.1 |
| 20.918 | 4.24339 | 100 |
| 22.484 | 3.95121 | 46.3 |
| 22.794 | 3.89818 | 18.8 |
| 23.971 | 3.70928 | 30.1 |
| 24.189 | 3.67645 | 12.5 |
| 24.587 | 3.61779 | 18.7 |
| 25.129 | 3.54094 | 9.4 |
| 25.826 | 3.44703 | 5.4 |
| 26.027 | 3.42076 | 5.3 |
| 26.459 | 3.3659 | 7.5 |
| 26.716 | 3.33408 | 9.4 |
| 27.222 | 3.27326 | 6.5 |
| 27.433 | 3.24856 | 6.4 |
| 27.827 | 3.20351 | 6 |
| 28.195 | 3.16245 | 5.8 |
| 28.475 | 3.13204 | 12.1 |
| 28.724 | 3.10542 | 5.7 |
| 29.041 | 3.07228 | 3.9 |
| 29.352 | 3.04039 | 5.8 |
| 30.285 | 2.94887 | 5.1 |
| 30.831 | 2.89788 | 6.7 |
| 31.238 | 2.86118 | 7.9 |
| 31.585 | 2.83041 | 5.1 |
| 31.959 | 2.7981 | 5.1 |
| 32.61 | 2.7437 | 3.6 |
| 32.994 | 2.71264 | 4.5 |
| 33.839 | 2.64682 | 4.6 |
| 34.058 | 2.63048 | 5.9 |
| 34.517 | 2.59639 | 7.9 |
| 35.351 | 2.537 | 4 |
| 35.798 | 2.50638 | 3.8 |
| 36.128 | 2.48418 | 3.4 |
| 36.455 | 2.4627 | 4.7 |
| 36.889 | 2.43469 | 3.6 |
| 38.094 | 2.36042 | 3.6 |
| 38.42 | 2.34108 | 4.7 |
| 38.759 | 2.32141 | 6.5 |

### 2. Differential Scanning Calorimetry (DSC):

Melting point was measured by differential scanning calorimetry (DSC) using a Parkin Elmer, Diamond DSC, the temperature gradient program is 50° C. to 260° C. at a ramp of 20° C. per min and sample mass of 1-2 mg.

The melting points were recorded for the crystalline form of Compound I, obtained using solvent of crystallisation selected from THF, 2-methyl tetrahydrofuran, a mixture of 2-methyl tetrahydrofuran and toluene, a mixture of 2-methyl tetrahydrofuran and heptane, methylethylketone, ethyl acetate or isopropyl acetate, and the melting points recorded were found to be identical, indicating that an identical crystalline form of Compound I was obtained with each of the above-mentioned solvents.

The melting temperature onset of the crystalline form of Compound I obtained using isopropyl acetate as solvent of crystallisation was determined to be 224.08±0.5° C. at 20 deg/min under nitrogen. The peak melting temperature was determined to be 226.83±0.5° C.

### Example 10

### (S)-2-Phenoxymethyloxirane (Compound A)

To a solution of NaOH (Merck, 159.99 g) in water (3.2 L), phenol (Spectrochem, 400 g, 4.211 mol) was added at room temperature and stirred for 10-15 min. To this reaction mixture was added R-epichlorohydrin (Frapps, 467.3 g, 5.053 mol) and tetrabutylammonium hydrogensulphate (Sisco, 8 g, 0.5797 mol) over a period of 10-15 minutes along with vigorous stirring by maintaining the temperature at room temperature (25-30° C.). The mixture was stirred for 3-3.5 h and on completion of the reaction, it was extracted with 1:1 ethyl acetate: petroleum ether (Commercial grade, 800 mL). The combined organic layer was dried over anhydrous sodium sulfate (Commercial grade, 200 g) and concentrated completely below 40° C. to afford the title compound A.

Yield range: 82-100%; G.C Purity: 70%; ¹H NMR (300 MHz, CDCl₃): δ 7.28-7.34 (m, 2H), 6.93-7.03 (m, 3H), 4.255 (m, 1H), 4.00 (m, 1H), 3.390 (t, 1H), 2.95 (m, 1H), 2.785 (m, 1H); MS: m/z 151 (M+H)⁺.

### Example 11

### N-Benzyl ethanolamine hydrogen sulphate (Compound C)

A solution of N-benzylethanolamine (A.K. Scientific, 1000 g, 6.6225 mol) in dichloromethane (Commercial grade, 6 L) was cooled to −5 to 0° C. Chlorosulphonic acid (Spectrochem, 771.5 g, 440.5 mL, 6.6255 mol) was added dropwise to the solution while maintaining the reaction temperature below 10° C. After addition was complete, the reaction mixture was then stirred at room temperature for 15-16 h. On completion of the reaction, ethanol (Commercial grade, 3 L) was added along with dichloromethane (3 L) and the reaction mixture was stirred at room temperature for 3-3.5 h. The solid obtained was filtered, washed with 1:1 EtOH:dichloromethane (Commercial grade, 2 L) and dried at 45-50° C. to afford the title compound C.

Yield range: 65-78%; HPLC purity: NLT 97%; LOD: NMT 2% w/w; ¹H NMR (300 MHz, D₂O): δ 7.388 (s, 5H), 4.214 (m, 4H), 3.32 (t, 2H); MS: m/z 232 (M+H)⁺.

### Example 12

### (S)-1-Benzyl-2-phenoxymethylmorpholine (Compound D)

To a solution of NaOH (Merck, 933 g, 23.33 mol) in water (1.75 L) which was cooled to 10-15° C., was added Compound C of example 11 (592.8 g, 2.566 mol) in portions while maintaining the reaction temperature at room temperature. A solution of Compound A of example 10 (350 g, 2.333 mol) in toluene (Commercial grade, 3.5 L) was added to the reaction mixture over 10-15 min, tetrabutylammonium hydrogensulphate (Sisco, 17.5 g) was added to the reaction mixture and the mixture was stirred at 45-50° C. for 15-16 h. On completion of the reaction, water (2.45 L) was added and the organic layer was separated. The organic layer was extracted with 10% aqueous HCl (3.5 L) twice. The combined aqueous layers were basified to pH of 9-10 with 10% NaOH solution (Merck, 3 L) and extracted with EtOAc (Commercial grade, 5.25+3.5 L). The combined organic layers were washed with water (3.5 L), 10% brine (3.5 L) and dried over anhydrous Na₂SO₄ (100 g). The solvent was removed completely by distillation below 50° C. to afford the title compound D as an oil.

Yield range: 68-90%; GC Purity: NLT 85%; ¹H NMR (300 MHz, CDCl₃): δ 7.33-7.23 (m, 7H), 6.96-6.93 (d, J=7.5 Hz, 1H), 6.90-6.88 (d, J=8.1 Hz, 2H), 4.05-3.90 (m, 4H), 3.77-3.66 (t, J=11.1 Hz, 1H), 3.55 (s, 2H), 3.49-2.86 (d, J=11.1 Hz, 1H), 2.70-2.66 (d, J=11.1 Hz, 1H), 2.274-2.187 (t, J=11.4 Hz, 1H), 2.131-2.063 (t, J=9.6 Hz, 1H), MS: m/z 284 (M+H)⁺.

### Example 13

### (S)-2-(Phenoxymethyl)morpholine (Compound E)

To a solution of compound D of example 12 (560 g, 1.978 mol) in methanol (Commercial grade, 5.6 L) in an atmosphere of CO₂ (obtained by adding small pieces of dry ice to the mixture) was added 10% Pd/C (Johnson M, 112 g). To the above reaction mixture was added ammonium formate (Avra, 560 g) at room temperature and the reaction mixture was heated at 60-65° C. for 1-2 h. On completion of the reaction, the reaction mixture is cooled to room temperature and was filtered through a celite bed, which was washed with MeOH (560 mL). The filtrate was concentrated completely below 45° C. The residue was dissolved in EtOAc (Commercial grade, 8.4 L) and the organic layer was washed with 10% brine (5.6 L) and dried over anhydrous Na₂SO₄. The organic layer was concentrated completely below 45° C. and the residue degassed below 45° C. to afford the title compound E.

Yield range: 74-90%; GC Purity: NLT 85%; Chiral HPLC: NLT 90% ee; ¹H NMR (300 MHz, CDCl₃) δ 7.31-7.26 (m, 2H), 6.99-6.91 (m, 3H), 4.11-4.09 (m, 2H), d 4.047-3.990 (m, 2H), 3.977-3.656 (t, 1H), 3.091-2.740 (m, 4H). MS: m/z 194 (M+H)⁺.

### Example 14

### In Vitro IGF-1R Kinase Assay

The in vitro kinase assays using IGF-1R kinase GST fusion proteins were conducted using a homogeneous time-resolved fluorescence (HTRF) format. Kinase reactions were carried out in a 384-well plate format in a final volume of 20 μL. The standard enzyme reaction buffer consisted of 50 mM Tris HCL (pH: 7.4), 1 mM EGTA, 10 mM MgCl₂, 2 mM DTT, 0.01% Tween-20, IGF-1R/IR kinase enzyme, poly GT peptide substrate (Perkin Elmer [Ulight Glu-Tyr (4:1)]n) and ATP [concentration equivalent to Kmₐₚₚ]. Compound I (methane sulfonate salt of (S)-ethyl 4-(2-carbamoyl-5-chloro-3-(2-(phenoxymethyl)morpholinosulfonyl)-1H-indol-7-ylamino) piperidine-1-carboxylate) in its amorphous and crystalline forms respectively in DMSO (<1%), were added to give a final inhibitor concentration ranging from 40 μM to 40 μM. Briefly, 2.5 μL enzyme and 2.5 μL inhibitor was pre-incubated for 10 minutes at 23° C. followed by the addition of 2.5 μL of poly GT substrate (final concentration of 50 nM). Reaction was initiated with the addition of 2.5 μL of ATP (final concentration of 20 μM for IGF-1R assay). After 1 hour incubation at 23° C., the kinase reaction was stopped with the addition of 5 μL EDTA (final concentration of 10 mM in 20 μL). Europium cryptate—labeled antiphosphotyrosine antibody PY20 (5 μL) was added (final concentration of 2 nM) and the mixture was allowed to equilibrate for 1 hour at 23° C. followed by reading the plate in an Envision plate reader. The intensity of light emission at 665 nm was directly proportional to the level of substrate phosphorylation. The IC₅₀ values for Compound I in both, the crystalline form and the amorphous form were determined by a four-parameter sigmoidal curve fit (Sigma plot or Graph pad) as represented in Table 2.

IGFRK enzyme used for the assay was intracellular kinase domain of human IGF-1R and expressed as GST fusion proteins using the baculovirus expression system and purified using glutathione—Sepharose column. IGFRK was used at a final concentration of 0.25 nM.

TABLE 2

| | Compound I | Compound I |
|---|---|---|
| | (crystalline | (amorphous |
| Enzyme | form) | form) |
| IGF-1R | 22 nM | 65.4 nM |

### Example 15

### IGF-1R Autophosphorylation Assay

Cells were grown and maintained in a medium containing 10% FBS. Cells grown as subconfluent monolayer, were subjected to serum starvation by replacing the respective culture medium with plain medium (containing no serum) and incubated for about 16 h at 37° C. in 5% CO₂ incubator. Serum starved cells were treated with compound I at different concentrations for 1 h at 37° C. in 5% CO₂ incubator and stimulated with IGF-1 (50 ng/mL) for the last 5 minutes of treatment with Compound I. After stimulation cells were washed twice with cold 1×PBS, pH 7.2 and cell lysates were prepared using CelLytic™ M cell lysis reagent (Sigma) containing protease and phosphatase inhibitors. Estimation of the total protein content in each cell lysate was carried out using Bradford reagent. Equal amount of protein from each lysate was subjected to Sodium Dodecyl Sulphate-Polyacrylamide Gel Electrophoresis (SDS-PAGE) followed by Western blotting protocol using specific antibody to pIGF-1R, IGF-1Rβ, pAkt, Akt, Pp70S6, p70S6, Pp44/42, p44/42 and loading control, β-actin. Imaging was done with BIO-RAD Image-Lab Software. The band density was then estimated using the software Image J. From the band densities of the various proteins at different concentrations of Compound I, the IC₅₀ concentrations for various proteins were calculated, and are enumerated in Table 3.

TABLE 3

| | pIGF-1R (nM) | | pAkt (nM) | | Pp70S6 (nM) | |
|---|---|---|---|---|---|---|
| Cell | Amorphous | Crystalline | Amorphous | Crystalline | Amorphous | Crystalline |
| line | form | form | form | form | form | form |
| Colo205 | 400 | 46 | 620 | 23 | — | — |
| A673 | <10 | 30 | 260 | 220 | 450 | — |
| MCF7 | 15 | 0.04 | 380 | 50 | 680 | 460 |

### Example 16

### Anti-Proliferative Assay

### Method for Measuring Cell Proliferation:

The cancer cell lines were seeded in triplicate (at density, from 3000-5000 cells/well depending on cell type) with 10% FCS in 180 μL of culture medium in tissue culture grade 96 well plates and allowed to recover for 24 h in humidified 5% CO₂ incubator at 37±1° C. After 24 h, media was replaced from the plate completely and 180 μL of fresh media containing 100 ng/mL IGF-1 without FCS was added followed with addition of 20 μL of 10× crystalline form of Compound I (dissolved first in DMSO and then in cell medium, final DMSO concentration did not exceed 0.5%) in wells. Compound I in crystalline form was used at concentration range of 0.1, 1, 3 and 10 μM and the plates were incubated for 72 h in humidified 5% CO₂ incubator at 37±1° C. Control wells were treated with vehicle (DMSO). At the end of the incubation periods, the plates were assayed by the CellTiter-Glo® Luminescent Cell Viability assay protocol. Percent cytoxicity was calculated at the various drug concentrations. Graph for cytotoxicity vs. concentration of Compound I was plotted, and the IC₅₀ values were determined

### CellTiter-Glo® Luminescent Cell Viability Assay

The CellTiter-Glo® Luminescent Cell Viability Assay is a homogeneous method to determine the number of viable cells in culture based on quantitation of the ATP present, which signals the presence of metabolically active cells. The amount of ATP is directly proportional to the number of cells present in culture

### Protocol

1. After 72 h incubation, the plate is equilibrated and its contents are maintained at room temperature for approximately 30 minutes.
2. A volume of CellTiter-Glo® Reagent was added in a volume equal to the volume of cell culture medium present in each well (e.g., 100 μL of reagent to 100 μL of medium containing cells for a 96-well plate).
3. The contents are mixed for 2 minutes on an orbital shaker to induce cell lysis. The plate is allowed to incubate at room temperature for 10 minutes to stabilize the luminescent signal. The luminescence is recorded using the POLARstar optima plate reader at excitation 536 nm and emission 590 nm
   Values are IC₅₀ in nM for different cell lines are provided for the crystalline form of Compound I in Table 4.

TABLE 4

| SK-ES | RD-ES | HCT116 | MCF7 | RD | A673 |
|---|---|---|---|---|---|
| 450 | 250 | 900 | 160 | 1400 | 1800 |

It should be noted that, as used in this specification and the appended claims, the singular forms “a”, “an”, and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred aspects and techniques. However, it should be understood that many variations and modifications may be made while remaining within the spirit and scope of the invention.

1-31. (canceled)

32. A stable crystalline form of (S)-ethyl 4-(2-carbamoyl-5-chloro-3-(2-(phenoxy methyl)morpholinosulfonyl)-1H-indol-7-ylamino) piperidine-1-carboxylate methane sulfonate (Compound I).

33. The crystalline form of Compound I according to claim 32, wherein said crystalline form is characterized by X-Ray diffraction peaks at an angle of refraction 2-theta of 9.22, 11.92, 13.58, 15.74, 18.37, 18.65, 18.95, 19.37, 19.59, 20.33, 20.92, 22.48, 22.79, 23.97, 24.19, 24.59, 28.48±0.2°.

34. The crystalline form of Compound I according to claim 32, wherein said crystalline form is characterized by a peak melting temperature of 226.83±0.5° C.

35. A process for the preparation of the crystalline form of Compound I as defined in claim 32, wherein said process comprises the steps of:
Step 1) purifying the free base of Compound I by treating a solution of said free base in a solvent selected from isopropyl acetate, tetrahydrofuran, 2-methyl tetrahydrofuran, toluene, heptane, methylethylketone, ethyl acetate, isopropyl acetate or a mixture thereof; with activated charcoal and Si-thiol (silicycle), filtering the resulting mixture through a celite bed, repeatedly washing the celite bed with the same solvent and evaporating the filtrate; and
Step 2) reacting the purified free base of compound I as obtained in Step (1) with methanesulfonic acid in said solvent at a temperature range of 70-80° C. for about 4-5 h to initiate crystallization of the Compound I as methane sulfonate salt, followed by cooling the resulting reaction mixture to room temperature and further to a temperature range of 0-5° C. to afford a crystalline mass, which is washed with the same chilled solvent to obtain the required crystalline form of Compound I.

36. A process for the preparation of the crystalline form of Compound I as defined in claim 32, comprising crystallizing an amorphous form of the compound I, (S)-ethyl 4-(2-carbamoyl-5-chloro-3-(2-(phenoxymethyl)morpholinosulfonyl)-1H-indol-7-ylamino) piperidine-1-carboxylate methane sulfonate) with a solvent selected from isopropyl acetate, tetrahydrofuran (THF), 2-methyl tetrahydrofuran, toluene, heptane, methylethylketone, ethyl acetate, isopropyl acetate or combinations thereof; wherein the amorphous form of compound I is obtained by reacting the free base of Compound I with methanesulphonic acid in tetrahydrofuran (THF) as solvent at room temperature (25-30° C.) for 30 min to 2 h.

37. The process according to claim 35, wherein said free base of Compound I is prepared by the steps comprising of:
step a: diazotising compound 1; 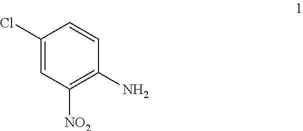 [SMILES: Nc1ccc(Cl)cc1[N+](=O)[O-]]
by reacting it with sodium nitrite (NaNO₂) and hydrochloric acid (HCl) at a temperature range of −10 to 5° C., followed by a dropwise addition of the diazotized mixture to an alkaline solution of ethyl 2-methyl-3-oxobutanoate in a base selected from sodium ethoxide (NaOEt), potassium hydroxide (KOH) or sodium hydroxide (NaOH) in a solvent selected from methanol or ethanol at a temperature range of −20° C. to −15° C. to obtain compound 2; 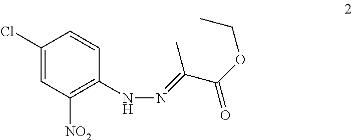 [SMILES: CCOC(=O)/C(C)=N/Nc1ccc(Cl)cc1[N+](=O)[O-]]
step b: cyclising the compound 2 by reaction with a Lewis acid selected from zinc chloride (ZnCl₂), aluminium chloride (AlCl₃), boron trifluoride (BF₃), phosphorus pentoxide (P₂O₅) or polyphosphoric acid at a temperature range of 80-120° C. for 5-12 h to obtain compound 3; 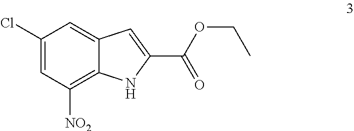 [SMILES: CCOC(=O)c1cc2cc(Cl)cc([N+](=O)[O-])c2[nH]1]
step c: sulphonating the compound 3 by reaction with sulphuric acid and acetic anhydride at a temperature range of 0-30° C. for 10-20 h to obtain compound 4; 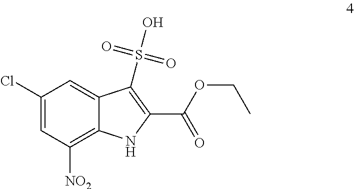 [SMILES: CCOC(=O)c1[nH]c2c([N+](=O)[O-])cc(Cl)cc2c1S(=O)(=O)O]
step d: reacting the compound 4 with oxalyl chloride or thionyl chloride in the presence of an organic base selected from triethylamine or pyridine in a solvent selected from N,N-dimethylformamide (DMF), methylene dichloride or a mixture thereof at a temperature range of 25-50° C. for 2-4 h to obtain the corresponding sulphonyl chloride 4A; 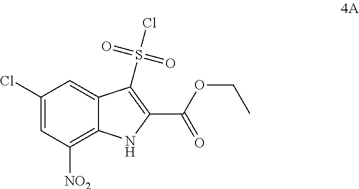 [SMILES: CCOC(=O)c1[nH]c2c([N+](=O)[O-])cc(Cl)cc2c1S(=O)(=O)Cl]
which is optionally isolated; and is then reacted with compound E: 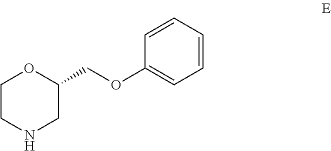 [SMILES: c1ccc(OC[C@@H]2CNCCO2)cc1]
in the presence of an organic base selected from pyridine or triethylamine in a solvent selected from dichloromethane or chloroform at room temperature (25-30° C.) for 1-4 h to obtain compound 5; 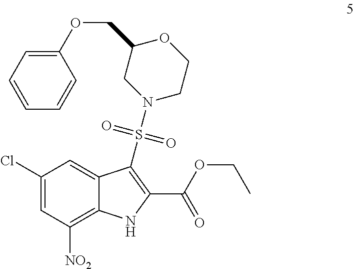 [SMILES: CCOC(=O)c1[nH]c2c([N+](=O)[O-])cc(Cl)cc2c1S(=O)(=O)N1CCO[C@H](COc2ccccc2)C1]
step e: reducing the compound 5 by reaction with a reducing agent selected from iron and ammonium chloride (Fe and NH₄Cl), zinc and hydrochloric acid (Zn and HCl) or stannous chloride (SnCl₂), for 2-8 h in a solvent selected from methanol, ethanol, THF, water or a mixture thereof, to obtain compound 6, which is purified using alcohol; 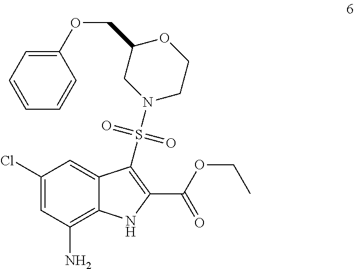 [SMILES: CCOC(=O)c1[nH]c2c(N)cc(Cl)cc2c1S(=O)(=O)N1CCO[C@H](COc2ccccc2)C1]
step f: reacting the compound 6 with isopropyl alcohol and ammonia at a temperature range of 80-120° C. at a pressure of 0.5-10 kg/cm² for 10-18 h in an autoclave or in a microwave for 10-15 min to obtain the compound 7, which is purified using alcohol; and 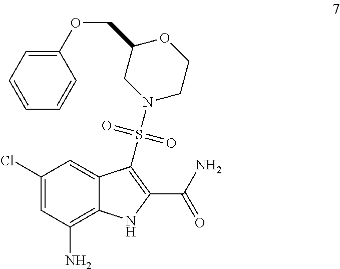 [SMILES: NC(=O)c1[nH]c2c(N)cc(Cl)cc2c1S(=O)(=O)N1CCO[C@H](COc2ccccc2)C1]
step g: reacting the compound 7 with the compound F: 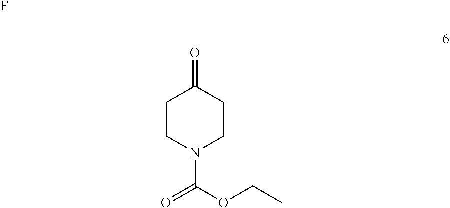
in the presence of trifluoroacetic acid in sodium triacetoxy borohydride as a base in a solvent selected from dichloromethane or ethyl acetate at room temperature (25-30° C.) for 0.5-2 h to obtain the compound I as a free base;

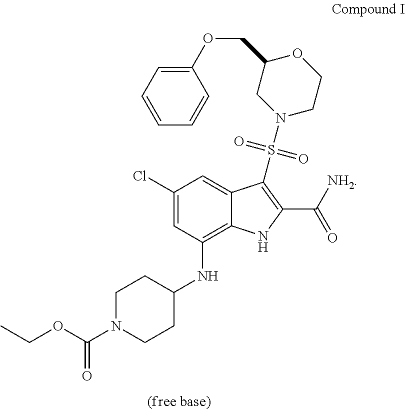 [SMILES: CCOC(=O)N1CCC(Nc2cc(Cl)cc3c(S(=O)(=O)N4CCO[C@H](COc5ccccc5)C4)c(C(N)=O)[nH]c23)CC1]

38. The process according to claim 37, wherein the preparation of compound E used in step d comprises the steps:
step a: reacting phenol with (R)-2-(chloromethyl)oxirane in the presence of a base selected from aqueous sodium hydroxide (NaOH) or aqueous potassium hydroxide (KOH) and tetrabutylammonium hydrogen sulphate as a phase transfer catalyst, at a temperature range of 80-120° C. for 1-4 h to obtain Compound A; 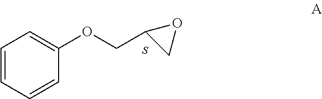
step b: reacting Compound B; 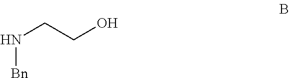 [SMILES: OCCNCc1ccccc1]
with chlorosulfonic acid in a solvent selected from chloroform, carbon tetrachloride, or dichloromethane, at 0-10° C. during addition of the acid over a period of 15-30 min, followed by at room temperature (25-30° C.) for 10-16 h to obtain Compound C; 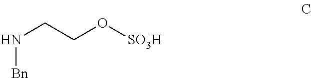 [SMILES: O=S(=O)(O)OCCNCc1ccccc1]
step c: reacting the Compound A obtained in step a with the Compound C obtained in step b in the presence of a base selected from aqueous sodium hydroxide (NaOH) or aqueous potassium hydroxide (KOH) in a solvent selected from toluene, dioxane or tetrahydrofuran (THF) in the presence of tetrabutylammoniun hydrogen sulfate as a phase transfer catalyst, at a temperature range of 30-50° C. for 10-16 h to obtain Compound D; and 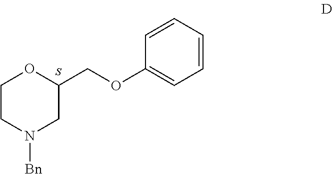
step d: carrying out debenzylation of Compound D by refluxing said Compound D with ammonium formate and 10% palladium over carbon (Pd/C) in an atmosphere of carbon dioxide in a solvent selected from ethanol or methanol at 50-70° C. for 1-3 h to obtain Compound E.

39. The process according to claim 35, wherein the solvent used in steps 1 and 2 is selected from tetrahydrofuran (THF), 2-methyl tetrahydrofuran, a mixture of 2-methyl tetrahydrofuran and toluene, a mixture of 2-methyl tetrahydrofuran and heptane, methylethylketone, ethyl acetate or isopropyl acetate.

40. The process according to claim 39, wherein the solvent is isopropyl acetate.

41. The process according to claim 37, wherein the base used in step a is sodium ethoxide (NaOEt).

42. The process according to claim 37, wherein in step b, when the cyclisation of the compound 2 is carried out using polyphosphoric acid as the Lewis acid, o-phosphoric acid is used as a solvent.

43. The process according to claim 37, wherein compound 4A of step d is isolated prior to reaction with the compound E.

44. A pharmaceutical composition comprising a therapeutically effective amount of crystalline form of Compound I according to claim 32 and a pharmaceutically acceptable excipient or a carrier.

45. A pharmaceutical composition comprising a therapeutically effective amount of crystalline form of Compound I according to claim 32 and a pharmaceutically acceptable carrier and other therapeutic agents.

46. A method for the treatment of diseases or disorders mediated by Insulin-Like-Growth Factor I Receptors or Insulin Receptors comprising administering to a subject in need thereof, a therapeutically effective amount of the crystalline form of Compound I according to claim 32.

47. The method according to claim 46, wherein the Insulin-Like-Growth Factor I Receptor and Insulin Receptor mediated disease or disorder is cancer.

48. The method according to claim 47, wherein the cancer is selected from astrocytoma, basal or squamous cell carcinoma, brain cancer, gliobastoma, bladder cancer, breast cancer, colon carcinoma, colorectal cancer, chrondrosarcoma, cervical cancer, adrenal cancer, choriocarcinoma, esophageal cancer, endometrial carcinoma, erythroleukemia, Ewing's sarcoma, gastrointestinal cancer, head and neck cancer, hepatoma, glioma, hepatocellular carcinoma, leukemia, leiomyona, melanoma, non-small cell lung cancer, neural cancer, ovarian cancer, pancreatic cancer, prostate cancer, renal cell carcinoma, rhabdomyosarcoma, small cell lung cancer, thymona, thyroid cancer, testicular cancer or osteosarcoma.

49. The method according to claim 48, wherein the cancer is selected from breast cancer, colon carcinoma, colorectal cancer, Ewing's sarcoma or rhabdosarcoma.
